(19) United States
(12) Patent Application Publication
(10) Pub. No.: US 2022/0355042 A1
(43) Pub. Date: Nov. 10, 2022

(54) PORTABLE ADJUSTABLE SYRINGE GUIDE AND VIAL HOLDER

(71) Applicants: Gail Winifred Nichols, Tamarac, FL (US); Richard Sutton Cherry, Tamarac, FL (US)

(72) Inventors: Gail Winifred Nichols, Tamarac, FL (US); Richard Sutton Cherry, Tamarac, FL (US)

(21) Appl. No.: 17/738,313

(22) Filed: May 6, 2022

Related U.S. Application Data
(60) Provisional application No. 63/186,011, filed on May 7, 2021.

(51) Int. Cl. A61M 5/315

(52) U.S. Cl. CPC A61M 5/31568; A61M 2005/3126

(57) ABSTRACT

A portable modular assistive device for storing, measuring, drawing and administering liquid injectable medication or other fluids from a vial and syringe, specifically to insulin for consumers and medical professionals in homes, while traveling and in medical facilities, having means to allow users to quickly, securely, easily and accurately measure and draw the desired amount of insulin and then administer the medication. The device is a portable syringe guide and vial holder comprising a horizontal elongated stabilizer made of rigid light-weight material with high tolerance and low friction, a rigid liquid medication vial protector at one end; a rigid movable syringe carriage with an opening on the top side to firmly hold the assembled syringe and needle to perfectly align the needle to the center section of the rubber top of the vial; a flexible expandable color-coded security strap; a flexible expandable color-coded resistance spacer ring, a removeable adjustable magnifier and calibrator bar, a rigid end cap at the other end of the horizontal stabilizer, with said holder having means to allow users to transport said holder in a pocket or pouch without risk of spillage from the vial, dislodging the components or damage to the vial, needle or syringe. Thus, a considerably more versatile, cost-effective, durable and efficient syringe guide and vial holder is provided that can accommodate a plurality of syringe and needle sizes as well as various liquid medications and other fluids in vials in the first embodiment as well as in other embodiments with ease of tracking quantities, contents, sizes and locations of a variety of vials, syringes and needles.

### CROSS-REFERENCE TO RELATED APPLICATIONS

This application claims the benefit of provisional patent application Ser. No. 63/186,011, filed May 7, 2021 by the present inventors

### FEDERALLY SPONSORED RESEARCH

Nonapplicable.

### SEQUENCE LISTING

Nonapplicable

### TECHNICAL FIELD

The present invention relates to assistive devices for administering injectable liquid medication, and more specifically to vials and syringes.

### BACKGROUND

Conventional methods of measuring and administering injectable liquid medication, particularly insulin, are inefficient, costly and inconvenient including insulin pen injectors and vials with insulin syringes and needles. The vial and syringe method requires the user to hold the vial with one hand while using their other hand to first insert the delicate needle into the small rubber top of the vial and then measure and draw insulin into the syringe, withdraw the needle from the vial, and administer the insulin.

The dexterity required for this process is affected by the user's health including sufficient vision to see the components, steady coordination of hands and eyes and mental clarity affected by stress and blood sugar level, amongst other factors which can result in costly damaged needles, inaccurate dosages, accidental needle sticks and frustration by users, particularly those with limited physical capabilities. Some users rely on caregivers to dispense insulin to themselves and their pets.

The ability for users to precisely control the penetration depth of the needle into the vial is especially advantageous given today's high cost of medicine and the consequent desire to avoid waste. The rubber top of the vial is squeezed into place by the vial manufacturer usually by a metal cap resulting in the outer circumference of the rubber being thicker and more difficult to pierce than the center, thus easily breaking delicate needles either by accidentally hitting the metal rim of the cap or the thicker rubber circumference. Therefore, center alignment of the needle in the rubber top must be virtually perfect.

Syringe is the most common form of insulin delivery, but there are other options, including insulin pens and pumps. Insulin pens can make taking insulin more convenient because they combine the medication and syringe in one unit. Unlike syringes, pens come preloaded with insulin including premixed insulin. Pens come in two basic types: disposable and reusable. Pen needles screw onto the top of an insulin pen and are generally reusable up to 5 times. Pens are more expensive than syringe, needle and vial.

Insulin pumps are more expensive than pens, but also more accurate and precise. The pumps deliver a constant flow of insulin throughout the day, allowing for a more flexible lifestyle. There are fewer needle pricks with insulin pumps.

So, manufacturers of insulin, syringes, needles and related medical devices have long sought means of accurately, cost-effectively and conveniently dispensing insulin to users in their homes, while traveling and in medical facilities.

### DESCRIPTION OF PRIOR ART

The following is a tabulation of some prior art that presently appears relevant:
U.S. Pat. No. 9,132,927 to Larson (2015)
U.S. Pat. No. 8,360,114 to Clark (2013)
U.S. Pat. No. 6,497,697 to Cohn (2002)
U.S. Pat. No. 6,006,798 to Lindquist (1999)
U.S. Pat. No. 5,247,972 to Tetreault (1993)
U.S. Pat. No. 4,489,766 to Montada (1984)
U.S. Pat. No. 3,602,272 to Stawski (1971)

While the need for efficient administration of liquid injectable medication such as insulin, has been facilitated by the use of syringes, especially by non-medically trained people, the prior art syringe guide and vial holders have left many areas available for improvement.

For example, many prior art syringe guide and vial holders fail to simplify the construction and use of syringe guide and vial holders (U.S. Pat. No. 8,360,114 to Clark (2013), U.S. Pat. No. 9,132,927 to Larson (2015), U.S. Pat. No. 6,497,697 to Cohn (2002), U.S. Pat. No. 5,247,972 to Tetreault (1993), U.S. Pat. No. 3,602,272 to Stawski (1971), and U.S. Pat. No. 4,489,766 to Montada (1984)). The resulting costly complex manufacturing process required to produce these prior art syringe guide and vial holders has limited their commercial availability.

Some prior art syringe guide and vial holders require a user to manipulate multiple retaining bands to secure and release a vial and a syringe (U.S. Pat. No. 6,006,798 to Lindquist (1999)). This prior art overcomplicates the use of syringe guide and vial holders for many syringe users who only need a simple device to help steady the vial and the needle during the insertion process.

In addition, the construction of some prior art syringe guide and vial holders has failed to allow precise positioning of a tip of a syringe needle into the reservoir of medicine in a vial (U.S. Pat. No. 9,132,927 to Larson (2015), U.S. Pat. No. 3,602,272 to Stawski (1971) and U.S. Pat. No. 4,489,766 to Montada (1984)). To aid in fully extracting the last contents in the vial, the needle should be inserted into the vial no further than the neck of the vial so that when the vial is inverted, as is typically done when filling a syringe, the needle will be immersed in the medicine regardless of the amount of medicine remaining in the vial.

In conclusion, insofar as we are aware, no portable syringe guide and vial holder formerly developed for administration of liquid injectable medication provides ease of use, adjustability and accuracy of dosage without the defects of limited portability, adjustability and secure positioning of the vial, needle and syringe.

### SUMMARY

In accordance with one embodiment, an improved portable assistive device for calibrating, measuring, drawing and extracting liquid injectable medication from a vial and syringe with needle, specifically to insulin for consumers and medical professionals in homes, while traveling and in medical treatment facilities comprises modular, adjustable rigid horizontal, curved, elongated and flexible supports having means to allow users to quickly, securely, easily and accurately measure and draw the desired amount of insulin into the syringe and needle and then administer the medication. As well, users can transport said device in a pocket or pouch without risk of spillage from the vial or damage to the vial, needle or syringe.

### Advantages:

Accordingly, several advantages of one or more aspects are as follows.
a) to provide an improved syringe guide and vial holder,
b) to provide a cost-effective device and method to measure and administer liquid injectable medication,
c) to provide means to facilitate extraction of liquids from medicine vials into syringes,
d) to provide means of increasing ease, convenience and accuracy to the user with limited manual dexterity and/or vision,
e) to provide means to intuitively accommodate inexperience by user in extracting liquid injectable medicines with a syringe,
f) to provide means to be held securely and accommodate easy use by each of a left-side and right-side human hand,
g) to provide means to be held comfortably by a human hand,
h) to provide means to be rotated, shaken or held in any position including upside down without components dislocating, breaking or spilling contents,
i) to provide means to re-use a medicine vial without removal of the vial from the syringe guide and vial holder,
j) to provide means to securely store a new or re-used syringe and needle,
k) to provide means to accurately position the needle on the syringe into the center of the rubber top of a medicine vial for easy extraction of liquid from the vial, and for storage between uses,
l) to provide means to easily emulsify cloudy insulin by rolling or rotating the syringe guide and vial holder,
m) to provide means to use most sizes of vial, insulin needle and syringe,
n) to provide means to store the syringe guide and vial holder in a transportable device,
o) to provide means to magnify the calibrations on the syringe for ease and accuracy of measuring the required dose,
p) to provide means to sterilize the vial cap without removal of the vial from the syringe guide and vial holder,
q) to provide means to position for use on a generally flat, horizontal surface without fear of tipping or rolling thus preventing subsequent damage to the components,
r) to provide means to be cleaned by hand or by a dishwashing machine,
s) to provide means to be encoded for easy identification of vial contents and size, needle size and syringe size, and
t) to provide means for a simple cost-effective design and resulting low-cost manufacturing process for improved opportunities for diabetics to benefit.

Thus, some embodiments provide a syringe guide and vial holder that is more efficient, cost-effective, durable and adaptable than prior-art syringe guide and vial holders. These and other benefits of one or more aspects will become apparent from a consideration of the ensuing description.

While the present invention has been described in terms of the preferred embodiments discussed in the above specification, it will be understood by one skilled in the art that the present invention is not limited to these particular preferred embodiments but includes any and all such modifications that are within the spirit and scope of the present invention as defined in the appended claims.

### BRIEF DESCRIPTION OF DRAWINGS

In the drawing, closely related figures have the same number but different alphabetic suffixes.

FIG. 1 schematically illustrates a right-side cross-sectional view of an assembled portable syringe guide and vial holder 10 constructed in accordance with the first embodiment

REFERENCE NUMERALS:

| | |
|---|---|
| 10 | syringe guide and vial holder assembled |
| 11 | vial protector |
| 11A | vial protector fixed slot |
| 11B | vial protector end flanges |
| 11C | vial protector viewing window |
| 12 | syringe carriage |
| 12A | carriage slot |
| 12B | carriage top opening |
| 12C | pivot post hole - lower |
| 13 | stabilizer bar end cap |
| 14A | vial security strap |
| 14B | resistance spacer ring |
| 15 | magnifier/calibrator |
| 15A | pivot post |
| 15B | pivot post hole - upper |
| 15C | calibrator bar |
| 15D | magnifier |
| 16 | stabilizer bar |

### DETAILED DESCRIPTION OF DRAWINGS—FIRST EMBODIMENT

In the drawing, closely related figures have the same number but different alphabetic suffixes.

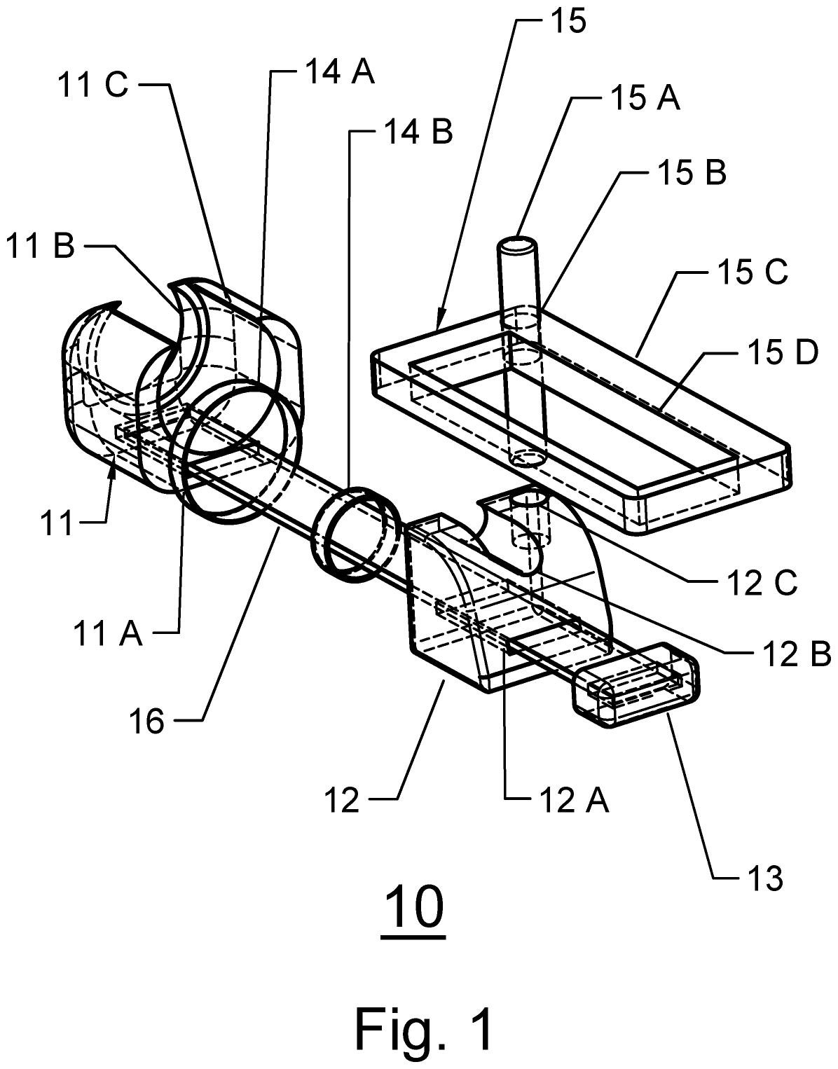

FIG. 1 schematically illustrates an overall cross-sectional view of the assembled syringe guide and vial holder 10 constructed in accordance with the first embodiment.

Holder 10 comprises modular interlocking components including: flat elongated horizontal rigid stabilizer bar 16, moveable curved rigid syringe carriage 12, fixed curved rigid vial protector 11, adjustable flexible vial security strap 14A, movable flexible resistance spacer ring 14B, fixed rigid stabilizer bar end cap 13, adjustable magnifier 15D and adjustable calibrator bar 15C.

Stabilizer bar 16 consisting of rigid material that is structurally strong, durable and can be corrosion resistant, that can be made of, but not limited to, metal such as steel and aluminum, bamboo and thermoplastic polymer material.

Stabilizer bar 16 interlocks with the rigid curved vial protector 11 at one end by means of the vial protector fixed slot 11A. Vial protector 11 also comprises vial protector end flanges 11B and vial viewing window 11C. The adjustable flexible vial security strap 14A is positioned around the neck or body of the vial (not shown). Stabilizer bar end cap 13 interlocks with the opposite end of the stabilizer bar 16.

Syringe carriage 12 interlocks with and moves slidingly from end to end on the rigid stabilizer bar 16 by means of carriage slot 12A having means to insert an assembled syringe and attached needle (not shown) in a resistance fit by means of a lengthwise top channel opening 12B. The movable flexible resistance spacer ring 14B is variably positioned between the syringe carriage 12 and vial protector 11.

Syringe carriage 12 interlocks with adjustable combined calibrator bar 15C and magnifier 15D having means to swivel horizontally and vertically on removable pivot post 15A that enters the upper pivot post hole 15B and extends into the lower pivot post hole 12C with resistance fit.

### Operation:

In operation, one uses the portable syringe guide and vial holder in the first embodiment to measure and draw the desired amount of insulin for administration to users in homes, medical facilities and while traveling. Thus, since the portable modular holder securely replaces the user's separate loose vial and assembled syringe with needle, it avoids the restrictions and inefficiencies of some prior-art syringe guide and vial holders.

If user has a new vial of medication with the protective outer cap in place, user removes the cap but not the rubber stopper.

User places the new or used vial inside the vial protector 11 interlocked at one end of the rigid elongated stabilizer bar 16, with the rubber stopper of the vial facing towards the movable syringe carriage 12.

User secures the vial in position within the vial protector 11 with the flexible expandable security strap 14A.

User slides the syringe carriage 12 to abut the stabilizer bar end cap 13. The interlocked stabilizer bar end cap 13 at the opposite end of the stabilizer bar 11 prevents the syringe carriage 12 from sliding off the horizontal stabilizer bar 11.

If the needle cover is still on the needle, user removes the cover. User positions the assembled syringe with exposed needle into the carriage top opening 12B with syringe needle flange abutting the front edge of the carriage top opening 12B and firmly presses down the flanged edge of the syringe to interlock with the syringe carriage 12 thereby creating a distinct snap acoustically and by touch to indicate the resistance fit of the syringe in the carriage top opening 12B and correct horizontal alignment of the assembled syringe and needle in the syringe carriage 12 slidingly secured on the upper side of the stabilizer bar 16 thereby positioning the syringe with needle pointing towards the vial protector 11 and the syringe plunger facing the stabilizer bar end cap 13. User rotates the barrel of the syringe, so calibrations are visible in the syringe carriage top opening 12B.

User variably positions the syringe carriage 12 containing the secured syringe and needle by means of the flexible expandable resistance spacer ring 14B to user's desired distance from the vial opening.

User sterilizes the rubber top of the vial usually with an alcohol swab just prior to measuring the insulin dose. User gently rotates the entire assembled holder 10 until all the white powder has dissolved in the insulin to emulsify it.

User pulls the plunger partially out of the syringe to draw air into the syringe and then slides the syringe carriage 12 towards the vial until the tip of the needle pierces the center of the rubber top of the vial and then pushes the syringe carriage 12 further toward the vial until the needle is inside the vial sufficiently to allow the user to push the plunger of the syringe to inject the air into the vial.

User positions the flexible resistance spacer ring 14B between the syringe carriage 12 and the vial protector 11 to desired location to enable user to extract the last vestiges of insulin from the vial, thereby reducing waste of insulin.

User positions the tip of the needle fully inside the insulin and then pulls the plunger to withdraw the correct dose of insulin into the syringe.

User can enhance view of syringe calibrations and amount of insulin in the syringe by swiveling the removable adjustable calibrator bar 15C and adjustable magnifier 15D horizontally and vertically on pivot post 15A that enters the upper pivot post hole 15B and extends into the lower pivot post hole 12C of the syringe carriage 12.

Before removing the needle from the vial, user checks the syringe for air bubbles. If bubbles are in the medicine, user rotates the vial and holder 11 further upright if necessary and taps the side of the syringe until the bubbles float to the top. User ejects the air with the plunger and draws insulin back into the syringe until measuring the correct dose.

User removes or swivels away the adjustable calibrator bar 15C and magnifier 15D on the pivot post 15A on the syringe carriage 12 so it is moved completely away from the syringe so the syringe can be freely removed by user for administration of the medication.

User slides the syringe carriage 12 away from the vial the full length of the stabilizer bar 16 until abutting the stabilizer bar end cap 13 so user can remove the syringe and needle from the syringe carriage 12 without touching the needle on anything.

User presses downward on the plunger end of the syringe to release it from the syringe carriage 12, thus creating a distinct audible and tangible snap, and thereby tilting the needle end of the syringe upwards. User entirely removes the syringe with needle from the syringe carriage 12. User is now ready to administer the medication.

After user administers the medication, user safely disposes the used needle and syringe and replaces them with new (recommended) or can choose to reuse the needle and syringe. User positions the new or re-used syringe and needle onto the syringe carriage 12 by means of the carriage top opening 12B.

User interlocks the calibrator bar 15C and magnifier 15D above the syringe on the syringe carriage 12 by means of the pivot post 15A inserted into the upper pivot post hole 15B extended into the lower pivot post hole 12C on the syringe carriage 12. User slides the syringe carriage 12 towards the vial so the needle is fully positioned inside the vial to protect the needle until next use.

The needle is held in position inside the vial by means of resistance fit of the rubber stopper as well as by position of the syringe on the syringe carriage 12 having resistance fit on the horizontal stabilizer bar 16.

User can carry the assembled syringe guide and vial holder 10 in their hand or by means of an optional pouch and sturdy case which further protects the holder 10 during storage and transportation.

As discussed above, the embodiments relate to a portable modular syringe guide and vial holder 10 for extracting liquid injectable medication from a vial and syringe, specifically to insulin medication for consumers and medical professionals in homes, while traveling and in medical treatment facilities having means to allow users to quickly, securely, easily and accurately measure insulin by sliding the syringe and needle in a movable syringe carriage 12 on a horizontal rigid stabilizer bar 16 to adjoin the rubber stopper of the vial held securely in a rigid vial protector 11 in order to fill the syringe with the required dose of insulin, and then sliding the syringe and needle back out to remove the syringe and needle from the holder 10 to administer the medication. As well, users can transport the device in a pocket or pouch without risk of spillage from the vial or damage to the vial, needle or syringe.

The portable syringe guide and vial holder 10 is an adjustable sturdy precision device offering consumers a practical solution to the considerable challenges experienced by the millions of diabetics taking insulin daily.

In one embodiment, the holder 10 is fabricated of a combination of durable light-weight plastic and metal materials, and may be 3-D rectangular in shape, measuring approximately 5 inches in length and 1 inch in width and 0.75 inches in depth.

There is a plurality of vial protector 11 versions available depending on user's preferred vial size and a plurality of syringe carriage 12 versions available depending on user's preferred size of needle (example 30 gauge, 1 cc, 5/16 th inch length and other sizes) and syringe (10 cc, 50 cc and other sizes) which affect the trajectory of the needle into the vial.

There is a plurality of colors available of flexible expandable vial security straps 14A and resistance spacer rings 14B to enable users to identify the specific size of vial and size of needle and syringe in the holder 10, of which user may own several depending on user requirements.

### Conclusion, Ramifications and Scope

The U.S. Centers for Disease Control and Prevention (CDC) recently published the National Diabetes Statistics Report (2020) with key findings including: 1) 34.2 million Americans—just over 1 in 10—have diabetes; 2) 88 million American adults—approximately 1 in 3—have prediabetes; 3) More people are developing type 1 and type 2 diabetes during youth, and racial and ethnic minorities continue to develop type 2 diabetes at higher rates. Likewise, the proportion of older people is increasing, and older people are more likely to have a chronic disease like diabetes. By addressing diabetes, many other related health problems can be prevented or delayed.

The Global Insulin Syringes Market size is expected to reach $2.1 billion by 2027, rising at a market growth of 4.5 percent compound annual growth rate (CAGR) during the forecast period. The insulin syringe market is classified into three categories based on syringe size: 3/10 CC Syringe (0.3 ml), 1/2 CC Syringe (0.5 ml), 1 CC Syringe (1 ml), and Others. In the year 2020, the 1 ml syringe size has taken up the majority of sales.

The world's aging population and expanding number of diabetes patients are two significant reasons driving the market's growth. Furthermore, rising obesity rates and individuals adopting unhealthy lifestyles contribute to market development. The growth in healthcare costs is a significant element driving demand for insulin syringes.

Diabetes can cause serious health complications including heart disease, blindness, kidney failure, and lower-extremity amputations. Diabetes is the seventh leading cause of death in the United States.

Type 1 diabetes, previously called insulin-dependent diabetes mellitus (IDDM) or juvenile onset diabetes, may account for 5 percent to 10 percent of all diagnosed cases of diabetes.

Type 2 diabetes was previously called non-insulin-dependent diabetes mellitus (NIDDM) or adult-onset diabetes. Type 2 diabetes may account for about 90 percent to 95 percent of all diagnosed cases of diabetes.

The CDC further states for Type 2 diabetes: “Treatment typically includes diet control, exercise, home blood glucose testing, and in some cases, oral medication and/or insulin. Approximately 40 percent of people with type 2 diabetes require insulin injections. How and when to take insulin is different for each person and can change over time. Syringes and insulin pens deliver insulin through a needle. You may take just one type of insulin or different types throughout the day depending on your lifestyle, what you eat, and your blood sugar levels.”

Conventional methods of dispensing liquid injectable medication, particularly insulin from vials using insulin syringes and needles are inefficient and inconvenient as they require the user to hold the vial with one hand while using their other hand to first insert the delicate needle into the small rubber top of the vial and then measure and draw insulin into the syringe, withdraw the needle from the vial, and administer the insulin. The dexterity required for this process is affected by the user's health including sufficient vision to see the components, steady coordination of hands and eyes and mental clarity affected by stress and blood sugar level, amongst other factors which can result in costly damaged needles, inaccurate dosages, accidental needle sticks and frustration by users particularly those with limited physical capabilities.

Although the description above contains many specifications, these should not be construed as limiting the scope of the assistive syringe guide and vial holder device but as merely providing illustrations of some of the present embodiments of the device. For example, in addition to insulin administration, the syringe guide and vial holder can be used with a plurality of sizes of vials, syringes and needles and a plurality of liquid injectable medications.

Thus, the scope of the syringe guide and vial holder should be determined by the appended claims and their legal equivalents, rather than by the examples given.

1. An article of portable liquid injectable medication storage and administration, comprising:
a. A rigid elongated horizontal stabilizer comprising a pre-determined cross-sectional shape including integrated support methods providing means for interlocking a rigid liquid medication vial protector at one end, and
b. Said protector having means to view vial contents, and
c. A flexible expandable security strap, and
d. A flexible expandable resistance spacer ring, and
e. A moveable syringe carriage providing means to interlock slidingly with the horizontal stabilizer while securely interlocking an assembled syringe with needle by means of an upper side opening with means to measure, calibrate and easily view contents of syringe, and
f. A rigid end cap at one end of the horizontal stabilizer which prevents said movable carriage from sliding off the horizontal stabilizer by means of an interlocking mechanism, and
g. An adjustable magnifier and calibration device to further assist in measuring the correct dose of medication.
Whereby (a) users of said portable syringe guide and vial holder device can quickly, securely, easily and accurately measure, draw and administer the desired amount of insulin or other medication or other fluids from the vial using an interlocking assembled syringe and needle, and (b) users can transport said syringe guide and vial holder device in a pocket or pouch without risk of spillage from the vial, dislodging of the components or damage to the vial, needle or syringe.

2. The article of portable liquid injectable medication administration in claim 1 wherein said rigid horizontal stabilizer can be made of, but is not limited to corrosion-resistant steel and aluminum material, bamboo and thermoplastic polymer material, including synthetic resin, polypropylene and polyester material.

3. The article of portable liquid injectable medication administration in claim 1 wherein said rigid protector for supporting said vial can be made of, but is not limited to, thermoplastic polymer material, including synthetic resin, polypropylene and polyester material.

4. The article of portable liquid injectable medication administration in claim 1 wherein said carriage for supporting said assembled syringe and needle can be made of, but is not limited to, thermoplastic polymer material, including synthetic resin, polypropylene and polyester material.

5. The article of portable liquid injectable medication administration in claim 1 wherein said end cap on the horizontal stabilizer can be made of, but is not limited to corrosion-resistant steel and aluminum material and thermoplastic polymer material, including synthetic resin, polypropylene and polyester material.

6. The article of portable liquid injectable medication administration in claim 1 wherein said flexible expandable security strap can be made of, but is not limited to, thermoplastic polymer material, including silicone, synthetic resin, polypropylene and polyester material.

7. The article of portable liquid injectable medication administration in claim 1 wherein said flexible expandable resistance spacer ring can be made of, but is not limited to, thermoplastic polymer material, including silicone, synthetic resin, polypropylene and polyester material.

8. The article of portable liquid injectable medication administration in claim 1 wherein said article has means to magnify and calibrate the liquid medication in the syringe.

9. The article of portable liquid injectable medication administration in claim 1 wherein said article has means to encode each of the medication vial, syringe and needle for visual and electronic identification of quantity, size, content and location.
